(19) United States
(12) Patent Application Publication
(10) Pub. No.: US 2011/0230439 A1
(43) Pub. Date: Sep. 22, 2011

(54) METHODS OF REDUCING ABSORPTION OF TRANS FATTY ACIDS USING WATER-SOLUBLE CELLULOSE DERIVATIVES

(76) Inventors: Kerr Anderson, Midlandmi, MI (US); David R. Albers, Midland, MI (US); Scott A. Young, Midland, MI (US); Wallace H. Yokoyama, Berkeley, CA (US)

(21) Appl. No.: 13/124,537

(22) PCT Filed: Oct. 16, 2009
(86) PCT No.: PCT/US09/60968

§ 371 Date: Jun. 1, 2011

Related U.S. Application Data
(60) Provisional application No. 61/106,167, filed on Oct. 17, 2008.

(51) Int. Cl. A61K 31/717; A61P 3/06

(52) U.S. Cl. 514/57

(57) ABSTRACT

Provided are food products comprising at least 0.5 g of trans fatty acid per serving and at least 1.33 weight percent of one or more water-soluble cellulose derivatives, based on the total weight of the food product, methods of ameliorating the harmful effects of trans fatty acids on a mammal that has consumed trans fatty acids, and methods of reducing the amount of trans fatty acids capable of being absorbed by a mammal ingesting a trans fatty acid containing food product.

### CROSS REFERENCE TO RELATED APPLICATIONS

This application claims the benefit of U.S. Provisional Application No. 61/106,167, filed Oct. 17, 2008.

### FIELD

The present invention relates to mammalian health and nutrition.

### STATEMENT REGARDING FEDERALLY SPONSORED RESEARCH

This invention was made under a Cooperative Research and Development Agreement with the U.S. Department of Agriculture, number 58-3K95-5-1072.

### BACKGROUND

Unlike other dietary fats, trans fatty acids are not essential for biological functions, nor are they healthful. To avoid confusion, naturally-occurring trans fat conjugated linoleic acid may offer health benefits, and so “trans fatty acids” typically refers to fatty acids containing one or more trans linkages that are not in a conjugated system.

Trans fatty acids only find their way into food by virtue of their formation in partially-hydrogenated vegetable oils. Trans fatty acids are by-products of partially-hydrogenating vegetable oils, which is done to increase their stability and shelf-life.

Putting aside their fat-based caloric contributions, trans fatty acids in foods have specifically come under strong criticism because trans fatty acids increase low density lipoprotein LDL (bad) cholesterol levels and decrease high density lipoprotein HDL (good) cholesterol levels in humans, increasing the risk of heart disease. Studies also link trans fatty acids to strokes and diabetes type 2. Health authorities recommend avoiding trans fatty acids, and some countries and at least one state in the United States have attempted to ban trans fatty acids in locally prepared foods. The United States Food and Drug Administration (FDA) has taken a different tack, requiring that manufacturers list trans fat on the Nutrition Facts panel of non-institutional foods when present at or over 0.5 g per serving, thus allowing consumers the choice to avoid trans fatty acids. This is not always practical, as partially-hydrogenated vegetable oils (and hence trans fatty acids) are often found in fast foods and snacks, which are highly desirable to even the most informed consumers.

Thus, what is needed are methods of reducing absorption of trans fatty acids by a mammal.

### SUMMARY

In one embodiment, the present invention provides a food product comprising at least 0.5 g of trans fatty acid per serving and at least 1.33 weight percent of one or more water-soluble cellulose derivatives, based on the total weight of the food product.

In another embodiment, the present invention provides a method of ameliorating the harmful effects of trans fatty acids on a mammal that has consumed trans fatty acids, comprising administering one or more water-soluble cellulose derivatives to the mammal.

In another embodiment, the present invention provides a method of reducing the amount of trans fatty acids capable of being absorbed by a mammal ingesting a trans fatty acid containing food product, comprising at least one of incorporating one or more water-soluble cellulose derivatives in the food product, or administering one or more water-soluble cellulose derivatives before, during, or after the consumption of the food product to the mammal.

### DETAILED DESCRIPTION

In one embodiment, the present invention provides a food product comprising at least 0.5 g of trans fatty acid per serving and at least 1.33 weight percent of one or more water-soluble cellulose derivatives, based on the total weight of the food product.

The term “trans fatty acids” refers to fatty acids containing one or more trans linkages that are not in a conjugated system. As mentioned above, trans fatty acids are an undesirable byproduct of partially-hydrogenated vegetable oils. Elaidic acid is a common trans fatty acid.

Two major avenues for incorporation of trans fatty acids into food involve the addition of margarine or vegetable shortening to the food or frying the food in partially hydrogenated oil. For purposes of this application, the food product may be any comestible typically prepared with partially hydrogenated vegetable oil. Examples of such foods include baked goods (including cookies, crackers, cakes, pies, muffins, donuts, and certain breads such as hamburger buns), deep-fried and pre-fried foods (including doughnuts, french fries, fried chicken, chicken nuggets, fish sticks, and taco shells), snack foods (including potato chips, corn chips, tortilla chips, peanut butter, whipped toppings, instant mashed potatoes, candy, and popcorn), and pre-packaged mixes (including cake mix, pancake mix, biscuit mix, cornbread mix, frosting mix, chocolate drink mix, certain frozen meals, pizza dough, ready to bake bakery products, toaster pastries, waffles, pancakes). Historically, fast food restaurants have been associated with trans fatty acids due to their use of pre-prepared foods and relatively low cost frying oils.

Although prior strategies for avoiding trans fatty acids have been to replace the partially-hydrogenated vegetable oils with costlier alternatives, it is now discovered that incorporation of water-soluble cellulose derivatives into a mammal's diet has a beneficial effect by reducing the amount of trans fatty acids absorbed by the mammal from ingesting the food. This does not appear to be a “bulk fiber effect,” as water-soluble cellulose derivatives were found to be far more efficacious than microcrystalline cellulose.

Food products of the present invention comprise at least 1.33 weight percent, preferably from 2 to 10 weight percent, more preferably from 2 to 6 weight percent of water-soluble cellulose derivative, based on the total weight of the food product. The most preferred percentage of water-soluble cellulose derivative in the food product depends on various factors mainly determined by nutritial and organoleptic considerations.

In one embodiment, the water-soluble cellulose derivative is a water-soluble cellulose ether or cellulose ester. The term “cellulose derivative” does not include unmodified cellulose itself which tends to be water-insoluble. The term “water-soluble” as used herein means that the cellulose derivative has a solubility in water of at least 2 grams, preferably at least 3 grams, more preferably at least 5 grams in 100 grams of distilled water at 25° C. and 1 atmosphere.

Preferred cellulose derivatives are water-soluble cellulose esters and cellulose ethers. Preferred cellulose ethers are water-soluble carboxy-C₁-C₃-alkyl celluloses, such as carboxymethyl celluloses; water-soluble carboxy-C₁-C₃-alkyl hydroxy-C₁-C₃-alkyl celluloses, such as carboxymethyl hydroxyethyl celluloses; water-soluble C₁-C₃-alkyl celluloses, such as methylcelluloses; water-soluble C₁-C₃-alkyl hydroxy-C₁₋₃-alkyl celluloses, such as hydroxyethyl methylcelluloses, hydroxypropyl methylcelluloses or ethyl hydroxyethyl celluloses; water-soluble hydroxy-C₁₋₃-alkyl celluloses, such as hydroxyethyl celluloses or hydroxypropyl celluloses; water-soluble mixed hydroxy-C₁-C₃-alkyl celluloses, such as hydroxyethyl hydroxypropyl celluloses, water-soluble mixed C₁-C₃-alkyl celluloses, such as methyl ethyl celluloses, or water-soluble alkoxy hydroxyethyl hydroxypropyl celluloses, the alkoxy group being straight-chain or branched and containing 2 to 8 carbon atoms. The more preferred cellulose ethers are methylcellulose, methyl ethyl cellulose, hydroxyethyl cellulose, hydroxypropyl cellulose, hydroxyethyl methylcellulose, hydroxypropyl methylcellulose, and carboxymethyl cellulose, which are classified as water-soluble cellulose ethers by the skilled artisans. The most preferred water-soluble cellulose ethers are methylcelluloses with a methyl molar substitution DS_{methoxyl} of from 0.5 to 3.0, preferably from 1 to 2.5, and hydroxypropyl methylcelluloses with a DS_{methoxyl} of from 0.9 to 2.2, preferably from 1.1 to 2.0, and a MS_{hydroxypropoxyl} of from 0.02 to 2.0, preferably from 0.1 to 1.2. The methoxyl content of methyl cellulose can be determined according to ASTM method D 1347 -72 (reapproved 1995). The methoxyl and hydroxypropoxyl content of hydroxypropyl methylcellulose can be determined by ASTM method D-2363-79 (reapproved 1989). Methyl celluloses and hydroxypropyl methylcelluloses, such as K100M, K250M, K4M, K1M, F220M, F4M and J4M hydroxypropyl methylcellulose are commercially available from The Dow Chemical Company. Combinations of two or more water-soluble cellulose derivatives are also useful.

The water-soluble cellulose derivative generally has a viscosity of from 5 to 2,000,000 cps (=mPa.s), preferably from 50 to 1,000,000 cps, more preferably from 1,000 to 500,000 cps, in particular from 10,000 to 300,000 cps, measured as a two weight percent aqueous solution at 20 degrees Celsius. The viscosity can be measured in a rotational viscometer.

Preferably, the water-soluble cellulose derivative is hydroxypropylmethyl cellulose, and more preferably, the water-soluble cellulose derivative is a hydroxypropyl methylcellulose with a DS_{methoxyl} of from 0.9 to 2.2, preferably from 1.1 to 2.0, and a MS_{hydroxypropoxyl} of from 0.02 to 2.0, preferably from 0.1 to 1.2.

In yet another embodiment, the present invention provides a method of ameliorating the harmful effects of trans fatty acids on a mammal that has consumed trans fatty acids, comprising administering one or more water-soluble cellulose derivatives to the mammal.

In yet another embodiment, the present invention provides a method of reducing the amount of trans fatty acids capable of being absorbed by a mammal ingesting a trans fatty acid containing food product, comprising at least one of incorporating one or more water-soluble cellulose derivatives in the food product, or administering one or more water-soluble cellulose derivatives before, during or after the consumption of the food product to the mammal.

The presence of one or more water-soluble cellulose derivatives in the food product or the administration of one or more water-soluble cellulose derivatives in combination with the food product is particularly effective. The water-soluble cellulose derivatives are preferably incorporated in such amount in the food product or are administered in such amounts before, during or after the consumption of the food product such that the daily dose of water-soluble cellulose derivative is generally in the range of 20 to 700 milligrams of water-soluble cellulose derivative per kilogram of mammal body weight per day. Preferably about 2 to 30, more preferably about 3 to 25 g of water-soluble cellulose derivative are ingested by a large mammal such as a human. The most preferred amount of water-soluble cellulose derivative depends on various factors, such as the fat content of the diet.

When the water-soluble cellulose derivative is administered separately from the food product, it can, for example be administered in the form of powdered, reverse-enteric coated or micro-encapsulated cellulose ether suspended in a flavored drink formulation, as tablets, capsules, sachets or caplets. When the water-soluble cellulose derivative is administered separately from the food product, it should be consumed in an appropriate timeframe with the food product, preferably within about 30 minutes before or after consumption of the food product, more preferably within about 15 minutes before or after consumption of the food product.

Preferably, the water-soluble cellulose derivative is hydroxypropylmethyl cellulose, and more preferably, the water-soluble cellulose derivative is a hydroxypropyl methylcellulose with a DS_{methoxyl} of from 0.9 to 2.2, preferably from 1.1 to 2.0, and a MS_{hydroxypropoxyl} of from 0.02 to 2.0, preferably from 0.1 to 1.2.

In one embodiment, the water-soluble cellulose derivative is present in an amount from at least 1.33 weight percent based on the total weight of the food product.

### EXAMPLES

The following examples are for illustrative purposes only and are not intended to limit the scope of the present invention. All percentages are by weight unless otherwise specified.

### Example 1

Male Syrian Golden hamsters with a starting body weight of between 80-90 grams (LVG strain, Charles River Laboratory, Willmington, Mass.) were fed standardized laboratory food (lab “chow”) for seven days to acclimate.

The acclimated hamsters were then assigned to one of the six groups (treatment or control in a specific diet group) described in TABLE 1:

TABLE 1

| | Supersize | | Pound Cake | | Pizza | |
|---|---|---|---|---|---|---|
| | Treat- | | Treat- | | Treat- | |
| Component | ment | Control | ment | Control | ment | Control |
| Supersize | 1700 | 1700 | — | — | — | — |
| Pound Cake | — | — | 480 | 480 | — | — |
| Pizza | — | — | — | — | 1250 | 1250 |
| Vitamin Mix | 17 | 17 | 5 | 5 | 13 | 13 |
| Mineral Mix | 60 | 60 | 17 | 17 | 46 | 46 |
| Corn oil | — | — | 10 | 10 | — | — |
| Casein | — | — | 58 | 58 | — | — |
| HPMC | 71 | — | 20 | — | 52 | — |
| MCC | — | 71 | — | 20 | — | 52 |

Components are listed in grams, with further details below:

Supersize—meals of hamburgers/french fries that were freeze dried and powdered. Energy intake was about 33.62%, and the trans fats content about 1.3%.

Pound Cake—freeze dried and powdered pound cake. Energy intake was about 32.8%, and the trans fats content about 0.7%.

Pizza—freeze dried and powdered pizza. Energy intake was about 26.5%, and the trans fats content about 0.3%.

Vitamin Mix—1.0 g/Kg Vitamin A Palmitate (500,000 IU/g), 0.6 g/Kg Vitamin D3 (400,000 IU/g), 10.0 g/Kg Vitamin E Acetate (500 IU/g), 10.0 g/Kg Inositol, 0.4 g/Kg Menadione Sodium Bisulfate, 9.0 g/Kg Niacin, 1.5 g/Kg Riboflavin, 2.0 g/Kg Thiamine HCl, 0.7 g/Kg Pyridoxine HCl, 4.0 g/Kg Calcium Pantothenate, 0.06 g/Kg Biotin, 0.2 g/Kg Folic Acid, 1.0 g/Kg Vitamin B12 (0.1%), and 959.54 g/Kg Sucrose. [From Recommendations by NRC Nutrient Requirements of Laboratory Animals, 3^{rd} Revised Edition, 1978. Dyets, Inc., Dr. H. L. Yowell]

Mineral Mix—388.2 g/Kg potassium phosphate, dibasic, 85.6 g/Kg calcium phosphate, dibasic, 363.9 g/Kg calcium carbonate, 109.0 g/Kg sodium choloride, 28.56 g/Kg magnesium oxide, 22.94 g/Kg ferric citrate, U.S.P., 0.06 g/Kg cobaltous carbonate, 0.29 g/Kg cupric carbonate, 0.01 g/Kg sodium fluoride, 0.06 g/Kg potassium iodide, 0.22 g/Kg manganese carbonate, 1.11 g/Kg zinc carbonate, 0.04 g/Kg chromium acetate, 0.01 g/Kg sodium selenite. [From Recommendations by NRC Nutrient Requirements of Laboratory Animals, 3^{rd} Revised Edition, 1978. Dyets, Inc., Dr. H. L. Yowell]

HPMC—hydroxypropyl methylcellulose (HPMC) having a methoxyl content of 19-24 percent, a hydroxypropoxyl content of 7-12 percent and a viscosity, measured as a two weight percent aqueous solution at 20° C., of about 250,000 mPa.s (cps). When present, the dosage was approximately 4% (wt/wt).

MCC—microcrystalline cellulose (MCC). When present, the dosage was approximately 4% (wt/wt).

The study was approved by the Animal Care and Use Committee, Western Regional Research Center, USDA, Albany, Calif., and all guidelines for the care and use of laboratory animals were followed. The animals were fed the diet from TABLE 1 assigned to them for 20 days, with collection of feces at 10 days and 20 days. The feces were freeze-dried at each collection time.

Feces were ground for 10 minutes on a SPEX 8000 or 80000 mixer mill using two tungsten carbide balls in a tungsten carbide cylinder. About 0.15g of ground fecal matter was weighed into a DIONEX 11 mL Accelerated Solvent Extractor (ASE) cell and combined with about 3.5 g diatomaceous earth (sand) with brief stirring. 100 μL of a 500 μg/mL solution of glyceryl trierucate in tetrahydrofuran (THF) was added to the cell, and the mixture was sandwiched between two cellulose filters. The cell contents were extracted with an extraction solvent containing 600 mL of hexane, 400 mL 2-propanol, and 20 mL acetic acid on a DIONEX ASE system with the following conditions: Preheat 1 min, pressure 2175 psi, heat 5 min, Temp 60° C., Static 10 min, flush 60%, purge 120 sec, cycle=2. This resulted in 20 mL of sample extract collected.

After mixing, 9.0 mL of extract was placed in a tared 16×125mm screw top culture tube. The extract was blown to dryness in a 45° C. water bath with a nitrogen purge. A 4 mL aliquot of acetonitrile was added and the mixture was blown to dryness again.

The samples were derivatized using the following procedure:

1. A 300 μL aliquot of 0.5N NaOH in MeOH (methanol) was added to each sample in the culture tube.

2. Tubes were capped, vortexed, and placed in a heating block at 100° C. for 5 min.

3. Samples were allowed to cool about 1 min.

4. Samples were uncapped and 350 μL of 14% boron trifluoride in MeOH was added.

5. Samples were capped, vortexed, and placed in a heating block at 100° C. for 5 min.

6. Samples were allowed to cool about 1 min.

7. Vials were uncapped and 2 mL heptane (0.200 mg/mL nonadecane in heptane) was added.

8. Vials were recapped, vortexed, and placed back in heating block at 100° C. for 5 min.

9. Samples were allowed to cool for about 1 min.

10. Vials were uncapped and 1 mL salt saturated H₂O solution was added.

11. They were recapped and placed on rocker for 5 min.

12. They were then centrifuged at 1500 rpm for 10 min.

13. Using Pasteur pipettes, about 1 mL of organic (top) layer was transferred into gas chromatography (GC) vials.

The derivatized samples were analyzed by gas chromatography with the following conditions:

Chromatograph: Agilent 6890 Series GC
Column: 60m ×0.25 mm (L×ID), 0.25 μm df,
Detector: FID (Flame Ionization Detector)

Temperatures:

Oven: 200° C. (5 min), to 250° C. at 5° C./min with 5 min. final hold time

Injector: 250° C.

Detector: 260° C.

Carrier: 3 mL/min at 200° C. (62.5 psi)

Split: 25 mL/min

Make-Up: Helium 27 mL/min

Air: 400 mL/min

Hydrogen: 30 mL/min

Sample Size: 2 μL

Data System: EZChrom Elite Version 3.2.1

For calibration, a spiking standard was prepared to contain 0.200 mg/mL nonadecane and 0.10 mg/mL methyl erucate in heptane. A calibration solution was prepared by weighing out 10 mg of NuCheck Standard 1A into a 1.0 mL volumetric flask. To this flask 110 μL of the spiking standard was added. The flask was diluted to volume with heptane containing 0.200 mg/mL nonadecane. NuCheck Standard 1A contained 20% each of methyl palmitate (C16:0), methyl stearate (C18:0), methyl oleate (C18:1), methyl linoleate (C18:2) and methyl linolenate (C18:3). Thus the calibration solution contained 2000 μg/mL of these five components along with 200 μg/mL nonadecane and 11 μg/mL methyl erucate.

Trans fatty acids were determined by summing the monounsaturated trans fatty acids C16:1, C18:1, C18:2 and C20:1. Trans fatty acids were identified by spiking experiments and by elution order provided in literature from the GC column manufacturer.

The results are listed in TABLE 2:

TABLE 2

| | Supersize | | Pound Cake | | Pizza | |
|---|---|---|---|---|---|---|
| | Day 10 | Day 20 | Day 10 | Day 20 | Day 10 | Day 20 |
| Treatment | 24* | 15* | 13 | 13* | 3.4* | 4.5* |
| Control | 2.5 | 4.2 | 6 | 5 | 0.4 | 0.6 |

Results are reported in mg/g. The treatment group (fed with 4% HPMC) had a significant increase in the excretion of trans fatty acids over the control group. Thus, HPMC facilitated the reduction of trans fatty acids being absorbed by an animal body. The data noted with an asterisk were significant at the 95% confidence interval.

### Example 2

Male Syrian Golden hamsters with a starting body weight of between 80-90 grams (LVG strain, Charles River Laboratory, Willmington, Mass.) were fed standardized laboratory food (lab “chow”) for seven days to acclimate.

The acclimated hamsters were then assigned to one of three diet groups described in TABLE 3:

TABLE 3

| | Bologna | | Cheese | | Potato Chip | |
|---|---|---|---|---|---|---|
| | Treat- | | Treat- | | Treat- | |
| Component | ment | Control | ment | Control | ment | Control |
| Bologna | 33 | 33 | — | — | — | — |
| Cheese | — | — | 35 | 35 | — | — |
| Potato Chip | — | — | — | — | 56 | 56 |
| Vitamin Mix | 1 | 1 | 1 | 1 | 1 | 1 |
| Mineral Mix | 3.5 | 3.5 | 3.5 | 3.5 | 3.5 | 3.5 |
| Corn oil | 2.5 | 2.5 | 2.5 | 2.5 | 2.5 | 2.5 |
| Casein | 10 | 10 | 8 | 8 | 12 | 12 |
| DL- | 0.3 | 0.3 | 0.3 | 0.3 | 0.3 | 0.3 |
| methionine | | | | | | |
| Cornstarch | 45.4 | 45.4 | 45.4 | 45.4 | 20.4 | 20.4 |
| Choline | 0.3 | 0.3 | 0.3 | 0.3 | 0.3 | 0.3 |
| Biturate | | | | | | |
| HPMC | 4 | — | 4 | — | 4 | — |
| MCC | — | 4 | — | 4 | — | 4 |

Components are listed in grams, with further details below:

Bologna—bologna that was freeze dried and powdered, having a trans fat content of about 1.0%.

Cheese—cheese that was freeze dried and powdered, having a trans fat content of about 0.4%.

Potato Chip—potato chips that were freeze dried and powdered, having a trans fat content of about 0.3%.

Vitamin Mix—1.0 g/Kg Vitamin A Palmitate (500,000 IU/g), 0.6 g/Kg Vitamin D3 (400,000 IU/g), 10.0 g/Kg Vitamin E Acetate (500 IU/g), 10.0 g/Kg Inositol, 0.4 g/Kg Menadione Sodium Bisulfate, 9.0 g/Kg Niacin, 1.5 g/Kg Riboflavin, 2.0 g/Kg Thiamine HCl, 0.7 g/Kg Pyridoxine HCl, 4.0 g/Kg Calcium Pantothenate, 0.06 g/Kg Biotin, 0.2 g/Kg Folic Acid, 1.0 g/Kg Vitamin B12 (0.1%), and 959.54 g/Kg Sucrose. [From Recommendations by NRC Nutrient Requirements of Laboratory Animals, 3^{rd} Revised Edition, 1978. Dyets, Inc., Dr. H. L. Yowell]

Mineral Mix—388.2 g/Kg potassium phosphate, dibasic, 85.6 g/Kg calcium phosphate, dibasic, 363.9 g/Kg calcium carbonate, 109.0 g/Kg sodium choloride, 28.56 g/Kg magnesium oxide, 22.94 g/Kg ferric citrate, U.S.P., 0.06 g/Kg cobaltous carbonate, 0.29 g/Kg cupric carbonate, 0.01 g/Kg sodium fluoride, 0.06 g/Kg potassium iodide, 0.22 g/Kg manganese carbonate, 1.11 g/Kg zinc carbonate, 0.04 g/Kg chromium acetate, 0.01 g/Kg sodium selenite. [From Recommendations by NRC Nutrient Requirements of Laboratory Animals, 3^{rd} Revised Edition, 1978. Dyets, Inc., Dr. H. L. Yowell]

HPMC—hydroxypropyl methylcellulose (HPMC) having a methoxyl content of 19-24 percent, a hydroxypropoxyl content of 7-12 percent and a viscosity, measured as a two weight percent aqueous solution at 20° C., of about 250,000 mPa.s (cps). When present, the dosage was approximately 4% (wt/wt).

MCC—microcrystalline cellulose (MCC). When present, the dosage was approximately 4% (wt/wt).

The study was approved by the Animal Care and Use Committee, Western Regional Research Center, USDA, Albany, Calif., and all guidelines for the care and use of laboratory animals were followed. The animals were fed the diet from TABLE 3 assigned to them, with collection of feces at 6 days.

Trans fatty acids content in the feces was analyzed as described in Example 1, and the results are listed in TABLE 4:

TABLE 4

| | Bologna | Cheese | Potato Chip |
|---|---|---|---|
| Treatment | 25* | 8* | 11* |
| Control | 13 | 2.8 | 1.5 |

Results are reported in mg/g. The treatment group (fed with 4% HPMC) had a significant increase in the excretion of trans fatty acids over the control group. Thus, HPMC facilitated the reduction of trans fatty acids being absorbed by an animal body. The data noted with an asterisk were significant at the 95% confidence interval.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A food product comprising:
at least 0.5 g of trans fatty acid per serving; and
at least 1.33 weight percent of one or more water-soluble cellulose derivatives, based on the total weight of the food product.

2. The food product of claim 1, wherein the food product comprises at least 3 weight percent of one or more water-soluble cellulose derivatives, based on the total weight of the food product.

3. The food product of claim 1, wherein the water-soluble cellulose derivative is a water-soluble cellulose ether or cellulose ester.

4. The food product of claim 1, wherein the water-soluble cellulose derivative is hydroxypropylmethyl cellulose.

5. The food product of claim 1, wherein the water-soluble cellulose derivative is a hydroxypropyl methylcellulose with a DS_{methoxyl} of from 0.9 to 2.2, preferably from 1.1 to 2.0, and a MS_{hydroxypropoxyl} of from 0.02 to 2.0, preferably from 0.1 to 1.2.

6. A method of ameliorating the harmful effects of trans fatty acids on a mammal that has consumed trans fatty acids, comprising:
administering one or more water-soluble cellulose derivatives to the mammal.

7. The method of claim 6, wherein the water-soluble cellulose derivative is a water-soluble cellulose ether or cellulose ester.

8. The method of claim 6, wherein the water-soluble cellulose derivative is hydroxypropylmethyl cellulose.

9. The method of claim 6, wherein the water-soluble cellulose derivative is a hydroxypropyl methylcellulose with a DS_{methoxyl} of from 0.9 to 2.2, preferably from 1.1 to 2.0, and a MS_{hydroxypropoxyl} of from 0.02 to 2.0, preferably from 0.1 to 1.2.

10. The method of claim 6, wherein the mammal is a human, and the water-soluble cellulose derivative is administered in an amount from about 2 to 30 g, more preferably about 3 to 25 g.

11. A method of reducing the amount of trans fatty acids capable of being absorbed by a mammal ingesting a trans fatty acid containing food product, comprising the steps of:
incorporating one or more water-soluble cellulose derivatives in the food product; or
administering one or more water-soluble cellulose derivatives before, during or after the consumption of the food product to the mammal.

12. The method of claim 11, wherein the water-soluble cellulose derivative is a water-soluble cellulose ether or cellulose ester.

13. The method of claim 11, wherein the water-soluble cellulose derivative is hydroxypropylmethyl cellulose.

14. The method of claim 11, wherein the water-soluble cellulose derivative is a hydroxypropyl methylcellulose with a DS_{methoxyl} of from 0.9 to 2.2, preferably from 1.1 to 2.0, and a MS_{hydroxypropoxyl} of from 0.02 to 2.0, preferably from 0.1 to 1.2.

15. The method of claim 11, wherein the water-soluble cellulose derivative is present in an amount from at least 1.33 weight percent based on the total weight of the food product.

16. The method of claim 11, wherein the water-soluble cellulose derivative is present in an amount from at least 3 weight percent based on the total weight of the food product.

17. The method of claim 11, wherein the mammal is a human, and the water-soluble cellulose derivative is administered in an amount from about 2 to 30 g, more preferably about 3 to 25 g.
